(19) United States
(12) Patent Application Publication
(10) Pub. No.: US 2007/0060610 A1
(43) Pub. Date: Mar. 15, 2007

(54) Methods and pharmaceutical formulations for increasing bioavailability

(76) Inventors: Michael F. Wempe, Kingsport, TN (US); Claus-Michael Lehr, Saarbrucken-Dudweiler (DE); Eva-Maria Collnot, Ottweiler (DE); Christiane Baldes, Saarbrucken (DE); Ulrich Friedrich Schafer, Sulzbach (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/453,068

(22) Filed: Jun. 15, 2006

Related U.S. Application Data
(60) Provisional application No. 60/691,102, filed on Jun. 16, 2005.

(51) Int. Cl. A61K 31/56; A61K 31/355; A61K 9/00

(52) U.S. Cl. 514/310; 424/400; 514/171; 514/458

(57) ABSTRACT

This invention relates to methods and compositions for increasing the bioavailability of pharmaceutical or lipophilic compounds. The invention further relates of methods of administering the compounds and compositions of the present invention to a human or animal.

This application claims the benefit of priority of U.S. Provisional Patent Application No. 60/691,102, filed Jun. 16, 2005.

Water-soluble vitamin E-active polyethylene glycol esters of tocopheryl acid such as succinates were developed to provide water-soluble molecules having high vitamin E activity via either oral or parenteral administration. Examples include the polyethylene glycol acid succinate of α-tocopherol, known as d-α-tocopheryl polyethylene glycol succinate (TPGS). U.S. Pat. No. 2,680,749 discloses TPGS molecules in which the polyethylene glycols have average molecular weights of 400, 1000, and those varying between 600 and 6000.

TPGS molecules where the polyethylene glycol (PEG) has an average molecular weight (MW) of about 1000 (TPGS 1000; available from Eastman Chemical Company, Kingsport, Tenn.) are currently used in oral pharmaceutical applications to enhance the bioavailability of various drugs. Due to the amphiphilic nature of TPGS 1000, incorporating TPGS 1000 into pharmaceutical formulations enhances oral bioavailability by solubilizing some hydrophobic drugs. TPGS 1000 is also believed to influence one or more transporter proteins, one example of which is P-glycoprotein (P-gp), an enzyme that acts as a cellular efflux pump. Therefore, TPGS 1000 may contribute to oral bioavailability enhancement by influencing efflux of some drugs. Additional modulators of cellular efflux pumps exist that are structurally distinct from TPGS.

Provided herein are methods of increasing the bioavailability of at least one pharmaceutical compound comprising administering at least one efflux inhibitor to a subject before administering the at least one pharmaceutical compound to the subject.

Also provided are methods of formulating a composition comprising combining at least one efflux inhibitor and at least one pharmaceutical compound, wherein the at least one efflux inhibitor is released from the composition before the at least one pharmaceutical compound when the composition is administered to a subject.

Further provided are compositions comprising at least one efflux inhibitor and at least one pharmaceutical compound, wherein the compositions are capable of releasing the at least one efflux inhibitor before the at least one pharmaceutical compound when the compositions are administered to a subject.

Additionally provided herein are methods of treating a subject in need of treatment with at least one pharmaceutical compound comprising administering at least one efflux inhibitor to the subject before administering the at least one pharmaceutical compound to the subject.

Also provided are methods of treating a subject in need of treatment with a pharmaceutical compound comprising administering the compositions of the present invention to the subject.

Further provided is an article comprising a composition comprising at least one efflux inhibitor and a composition comprising at least one pharmaceutical compound wherein, upon oral administration to a human or mammal, the article releases the at least one efflux inhibitor into the gut of the human or mammal before the composition releases the at least one pharmaceutical compound into the gut of the human or mammal.

Additionally provided is a kit comprising a composition comprising at least one efflux inhibitor, a composition comprising at least one pharmaceutical compound, and instructions for administration of both the composition comprising at least one efflux inhibitor and the composition comprising at least one pharmaceutical compound to a human or mammal in a manner that will result in the at least one efflux inhibitor contacting selected cells, tissues or organs for a selected period of time before the at least one pharmaceutical compound contacts the selected cells, tissues or organs.

Also provided is a container, wherein the contents of the container comprise at least one pharmaceutical compound and at least one efflux inhibitor, wherein the container contains, is labeled, or is otherwise accompanied by instructions for administration of both the at least one efflux inhibitor and the at least one pharmaceutical compound to a human or mammal in a manner that will result in the at least one efflux inhibitor contacting selected cells, tissues or organs for a selected period of time before the at least one pharmaceutical compound contacts the selected cells, tissues or organs.

Additional embodiments of the invention are set forth in the description which follows, or may be learned by practice of the invention.

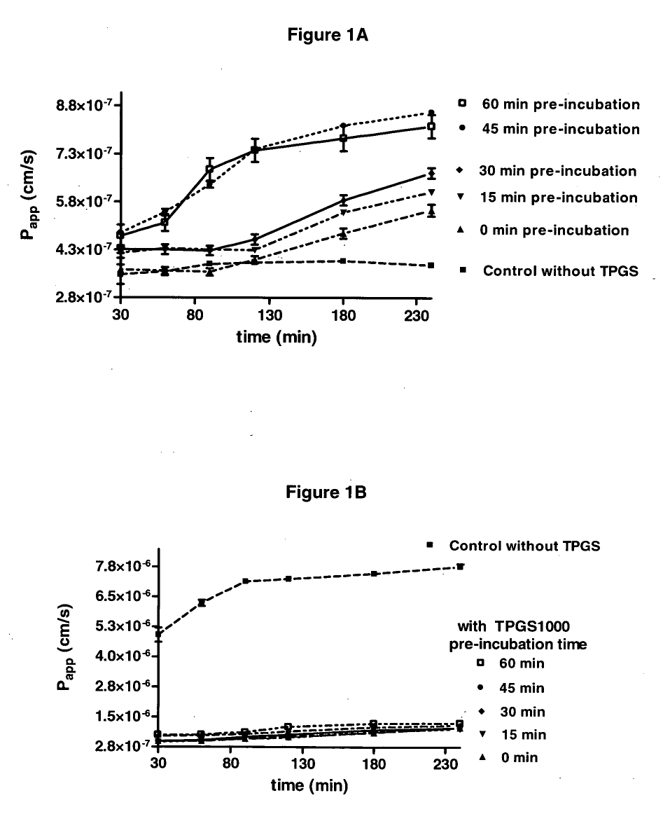

FIG. 1A and 1B illustrate Rhodamine 123 (13 μM) time-course transport across Caco-2 monolayers in the presence of TPGS 1000 (33 μM) on both the apical and basolateral sides using different pre-incubation times; 1A: absorptive transport, Ap→Bl; 1B: secretory transport, Bl→Ap; mean ±SEM, n=12.

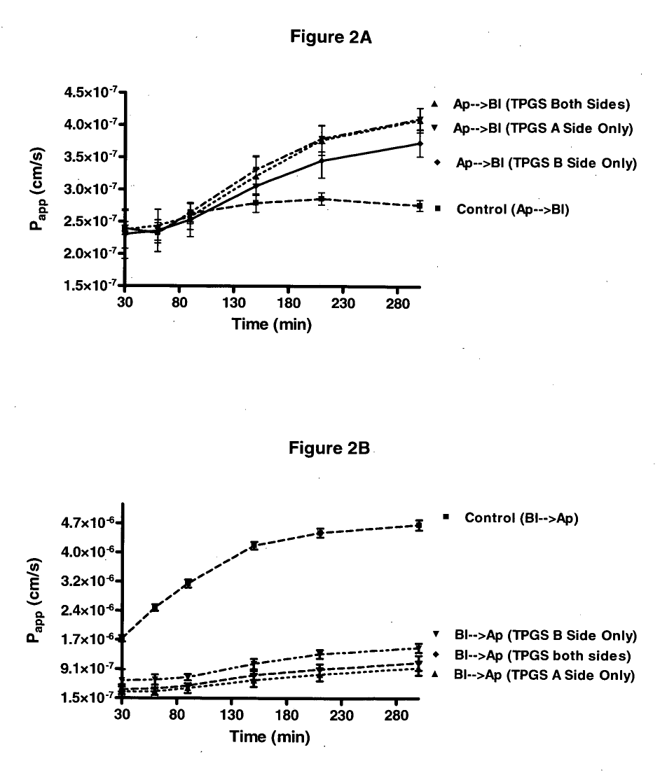

FIG. 2A and 2B illustrate Rhodamine 123 (13 μM) time-course transport across Caco-2 monolayers in the presence of TPGS 1000 (33 μM) on apical, basolateral, or both sides with pre-incubation without TPGS 1000; 2A: absorptive transport, Ap→Bl; 2B: secretory transport, Bl→Ap; mean ±SEM, n=3.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used throughout this application, the term molecular weight, including the abbreviation MW, shall refer, in connection with a single molecule, to the molecular weight of that molecule. With respect to a poly-disperse preparation containing polymer molecules of differing molecular weights, molecular weight shall refer to weight-average molecular weight (M_{W}).

“Disease” refers to any disease, disorder, condition, symptom, or indication.

The term “pharmaceutical compound,” “pharmaceutical,” “compound for pharmaceutical use” or “drug” refers to any substance which, when administered to a human or animal under conditions effective to cause a therapeutic or prophylactic effect. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfarim and disulfarim-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitors.

The term “increasing bioavailability” or “increased bioavailability” of a pharmaceutical or lipophilic compound means that the administration of a efflux inhibitor prior to the pharmaceutical or lipophilic compound results in an increase in the portion of the dose of the pharmaceutical or lipophilic compound that reaches one or more targeted systemic fluids, organs, tissues or cells as compared to administration of the pharmaceutical or lipophilic compound at the same time as the efflux inhibitor. In vitro and in vivo assays known in the art may be used to assess the relative bioavailability of a pharmaceutical or lipophilic compound when the efflux inhibitor is administered prior to, or at the same time as, the pharmaceutical or lipophilic compound. For example, in vitro assays employing Caco-2 cells such as those discussed below and in vivo animal studies may be used.

Increased bioavailability can include any mechanism that that has a desired effect on cellular efflux, cellular influx, or clearance. “Clearance” includes any type of elimination of one or more compounds from cells, blood, plasma, tissues or organs (e.g. intestinal clearance, hepatic clearance, renal clearance, and pulmonary clearance each describe elimination of compounds from the blood). Clearance may be described via the observed differences of renal excretion and elimination by all other processes including influx and efflux mechanisms (e.g. gastrointestinal clearance, excretory clearance, biliary clearance and enterohepatic cycling, metabolic clearance). Examples of systemic fluids include, but are not limited to: blood; cerebrospinal fluid; lymph; and any other tissue fluids (including increased amounts in tissues that are bathed by such fluids, such as the brain, tissue of one or more visceral organs, connective tissue, muscle, fat, or one or more tissues in the skin). In. some embodiments, the increase is systemic, as in the case of an increase measurable anywhere in the blood. In some embodiments, the increase is more localized, as is the case with some embodiments involving topical administration in which the increase is measured only in areas near the administration. An increase in portion of the dosage that reaches a fluid or tissue measurable by any reliable means is within this definition, including but not limited to increases identified by measuring the total systemic drug concentration over time after administration. In some embodiments, concentrations are determined by measuring the tissue or fluids themselves, or by measuring fractions thereof (for example, without limitation, serum or plasma in the case of blood). In some embodiments, increases for compounds that are excreted metabolized and/or un-metabolized in urine are determined by measuring levels of compounds or metabolites of the compounds in urine and will reflect an increase in systemic concentrations. In some embodiments an increase in compound bioavailability is defined as an increase in the Area Under the Curve (AUC). AUC is an integrated measure of systemic compound concentrations over time in units of mass-time/volume and is measured from the time compound is administered (time zero) to 12 to 24 hours, then is extrapolated to infinity (when no compound(s) remaining in the body can be measured). Information regarding monitoring substances within a subject are known to persons of ordinary skill in the art and may be found in references such as M. Rowland and T. N. Tozer, Clinical Pharmacokinetics Concepts and Applications (third Ed., 1995), Lippincott Willams and Wilkins, Philadelphia.

The term “efflux inhibitor” refers to an agent that is capable of causing efflux inhibition. The term “efflux inhibition” refers to a reduction of the transport of a compound in the basolateral to apical (Bl-Ap) direction or a reduction in the ratio of the transport of a compound in the basolateral to apical (Bl-Ap) direction to the transport of the compound in the apical to basolateral (Ap-Bl) direction.

As used throughout this application, the term “lipophilic compounds” shall mean compounds having solubility in water that is in the “sparingly soluble” range, or lower. (Persons of ordinary skill in the art will understand that, for compounds that are “sparingly soluble in water,” the quantity of water needed to dissolve one gram of the compound will be in the range beginning at about 30 mL and ending at about 100 mL. Compounds having solubility lower than “sparingly soluble” in water will require greater volumes of water to dissolve the compounds).

The term “lipophilic compound for pharmaceutical use” or “pharmaceutical compound that is a lipophilic compound” refers to a lipophilic compound that is also a compound for pharmaceutical use. Examples of lipophilic compounds for pharmaceutical use include, but are not limited to, itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene and ritonavir.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to any of a diluent, adjuvant, excipient or carrier with which at least one compound of the present disclosure is administered.

“Subject” includes mammals and humans. The term encompasses cells derived from a subject as well as the organism as a whole.

“Therapeutically effective amount” or “effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying or preventing the onset or reoccurence of the disease or disorder or at least symptoms thereof in a subject which may be exposed to or predisposed to or may have previously suffered from a disease or disorder even though that subject does not yet experience or display symptoms of the disease or disorder.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

The present invention is directed in part to methods of increasing the bioavailability of a pharmaceutical or lipophilic compound by administering a efflux inhibitor to a subject followed by administering the pharmaceutical or lipophilic compound to the subject. In some embodiments, the pharmaceutical compound is a lipophilic compound for pharmaceutical use.

With the invention, the absorptive transport of a pharmaceutical or lipophilic compound can be increased when a cell, tissue or subject is first contacted with a efflux inhibitor for an effective time before being contacted with the pharmaceutical or lipophilic compound. In certain embodiments, the efflux inhibitor inhibits secretory transport in addition to increasing the absorptive transport of the pharmaceutical or lipophilic compound.

Any pharmaceutical or lipophilic compound known in the art may be used in the compositions or methods of the present invention. Exemplary pharmaceutical or lipophilic compounds include itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene, erythromycin, clarithromycin, carbamazepine, ketoconazole, indinavir, progesterone, ritonavir, amiodarone, atorvastatin, azithromycin, carvedilol, chlorpromazine, cisapride, ciprofloxacin, cyclosporine, dapsone, diclofenac, diflunisal, flurbiprofen, glipizide, glyburide, griseofulvin, indomethacin, lansoprazole, mebendazole, naproxen, warfarin, terfenadine, talinolol, sirolimus, piroxicam, phentoin, domperidone, and oxaprozin.

Any efflux inhibitor known in the art may be used in the compositions or methods of the present invention. Suitable efflux inhibitors include compounds that inhibitor cellular efflux pumps such as P-gp. Examples of efflux inhibitors include aryl-indane compounds such as those disclosed in U.S. Patent Publication No. US 2002/0128231; TPGS compounds; cyclosporine compounds; and the inhibitor compounds XR9576, GF120918, T101933, OC144-093, LY335979, PSC833, XR9051 and VX-710 (See, e.g., Mistry et al., Cancer Research 61:749-758, 2001).

In certain embodiments, the efflux inhibitor may be a TPGS compound. TPGS compounds suitable for use in the present invention include any TPGS compound or analog thereof known in the art. Examples include, but are not limited to, TPGS 1000, TPGS compounds with PEG molecular weights ranging from 200-40,000, or TPGS analog compounds. TPGS compounds of varying molecular weights and methods of their production are disclosed in U.S. application Ser. No. 11/238,439, filed Sep. 29, 2005. TPGS analog compounds and methods of their production are disclosed in U.S. Provisional Application No. 60/788,053, filed Apr. 3, 2006.

In certain embodiments, the TPGS compounds may contain a polyethylene glycol (PEG) that has a selected molecular weight or is within a selected range of molecular weights. In some embodiments, the polyethylene glycol molecular weight is less than or equal to about 900. In some embodiments, the polyethylene glycol molecular weight is less than or equal to about 800. In some embodiments, the polyethylene glycol molecular weight is less than or equal to about 700. In some embodiments, the polyethylene glycol molecular weight is less than or equal to about 600. In some embodiments, the polyethylene glycol molecular weight is less than or equal to about 500. In some embodiments, the polyethylene glycol molecular weight is less than or equal to about 400. In some embodiments, the polyethylene glycol molecular weight is less than or equal to about 300. In some embodiments, the polyethylene glycol molecular weight is less than or equal to about 200. In some embodiments, the polyethylene glycol molecular weight is greater than or equal to about 1500. In some embodiments, the polyethylene glycol molecular weight is greater than or equal to about 1600. In some embodiments, the polyethylene glycol molecular weight is greater than or equal to about 1700. In some embodiments, the polyethylene glycol molecular weight is greater than or equal to about 1800. In some embodiments, the polyethylene glycol molecular weight is greater than or equal to about 1900. In some embodiments, the polyethylene glycol molecular weight is less than or equal to about 2000. In some embodiments, the polyethylene glycol molecular weight is greater than or equal to about 2100. In some embodiments, the polyethylene glycol molecular weight is greater than or equal to about 2200. In some embodiments, the polyethylene glycol molecular weight is greater than or equal to about 2300. In some embodiments, the polyethylene glycol molecular weight is greater than or equal to about 2400. In some embodiments, the polyethylene glycol molecular weight is greater than or equal to about 2500. In some embodiments, the polyethylene glycol molecular weight is greater than or equal to about 2600. In some embodiments, the polyethylene glycol molecular weight is less than or equal to about 3000. In some embodiments the polyethylene glycol molecular weight is greater than or equal to 3350. In some embodiments, the polyethylene glycol molecular weight is greater than or equal to about 3500. In some embodiments, the polyethylene glycol molecular weight is less than or equal to about 4000. In some embodiments, the polyethylene glycol molecular weight is greater than or equal to about 4500. In some embodiments, the polyethylene glycol molecular weight is less than or equal to about 5000. In some embodiments, the polyethylene glycol molecular weight is greater than or equal to about 5500. In some embodiments, the polyethylene glycol molecular weight is less than or equal to about 6000. In some embodiments, the polyethylene glycol molecular weight is less than or equal to about 7000. In some embodiments, the polyethylene glycol molecular weight is less than or equal to about 8000. In some embodiments, the polyethylene glycol molecular weight is less than or equal to about 9000. In some embodiments, the polyethylene glycol molecular weight is less than or equal to about 10000. In some embodiments, the polyethylene glycol molecular weight is less than or equal to about 11000. In some embodiments, the polyethylene glycol molecular weight is less than or equal to about 12000. In some embodiments, the polyethylene glycol molecular weight is less than or equal to about 13000. In some embodiments, the polyethylene glycol molecular weight is less than or equal to about 14000. In some embodiments, the polyethylene glycol molecular weight is less than or equal to about 15000. In some embodiments, the polyethylene glycol molecular weight is less than or equal to about 16000. In some embodiments, the polyethylene glycol molecular weight is less than or equal to about 17000. In some embodiments, the polyethylene glycol molecular weight is less than or equal to about 18000. In some embodiments, the polyethylene glycol molecular weight is less than or equal to about 19000. In some embodiments, the polyethylene glycol molecular weight is less than or equal to about 20000. In some embodiments, the polyethylene glycol molecular weight is less than or equal to about 22000. In some embodiments, the polyethylene glycol molecular weight is less than or equal to about 24000. In some embodiments, the polyethylene glycol molecular weight is less than or equal to about 26000. In some embodiments, the polyethylene glycol molecular weight is less than or equal to about 28000. In some embodiments, the polyethylene glycol molecular weight is less than or equal to about 30000. In some embodiments, the polyethylene glycol molecular weight is less than or equal to about 32000. In some embodiments, the polyethylene glycol molecular weight is less than or equal to about 34000. In some embodiments, the polyethylene glycol molecular weight is less than or equal to about 36000. In some embodiments, the polyethylene glycol molecular weight is less than or equal to about 38000. In some embodiments, the polyethylene glycol molecular weight is less than or equal to about 41000. Embodiments also exist in which the. molecular weight of the polyethylene glycol is in specific ranges, for example 50-150, 100-200, 150-250, 200-300, 250-350, 300-400, 350-450, 400-500, 450-550, 500-600, 550-650, 600-700, 650-750, 700-800, 750-850, 800-900, 850-950, 1000-1100, 1050-1150, 1100-1200, 1150-1250, 1200-1300, 1250-1350, 1300-1400, 1350-1450, 1400-1500, 1450-1550, 1500-1600, 1550-1650, 1600-1700, 1650-1750, 1700-1800, 1750-1850, 1800-1900, 1850-1950, 1900-2000, 1950-2050, 2000-2100, 2050-2150, 2100-2200, 2150-2250, 2200-2300, 2250-2350, 2300-2400, 2350-2450, 2400-2500, 2450-2550, 2500-2600, 2550-2650, 2600-2700, 2650-2750, 2700-2800, 2750-2850, 2800-2900, 2850-2950, 2900-3000, 2950-3050, 3000-3100, 3050-3150, 3100-3200, 3150-3250, 3200-3300, 3250-3350, 3300-3400, 3350-3450, 3400-3500, 3450-3550, 3500-3600, 3550-3650, 3600-3700, 3650-3750, 3700-3800, 3750-3850, 3800-3900, 3850-3950, 3900-4000, 3950-4050, 4000-4100, 4050-4150, 4100-4200, 4150-4250, 4200-4300, 4250-4350, 4300-4400, 4350-4450, 4400-4500, 4450-4550, 4500-4600, 4550-4650, 4600-4700, 4650-4750, 4700-4800, 4750-4850, 4800-4900, 4850-4950, 4900-6000, 4950-6050, 5000-5100, 5050-5150, 5100-5200, 5150-5250, 5200-5300, 5250-5350, 5300-5400, 5350-5450, 5400-5500, 5450-5550, 5500-5600, 5550-5650, 5600-5700, 5650-5750, 5700-5800, 5750-5850, 5800-5900, 5850-5950, 5900-6000, 5950-6050, 6000-6100, 6050-6150, 6100-6200, 6150-6250, 6200-6300, 6250-6350, 6300-6400, 6350-6450, 6400-6500, 6450-6550, 6500-6600, 6550-6650, 6600-6700, 6650-6750, 6700-6800, 6750-6850, 6800-6900, 6850-6950, 6900-7000, 6950-7050, 7000-7100, 7050-7150, 7100-7200, 7150-7250, 7200-7300, 7250-7350, 7300-7400, 7350-7450, 7400-7500, 7450-7550, 7500-7600, 7550-7650, 7600-7700, 7650-7750, 7700-7800, 7750-7850, 7800-7900, 7850-7950, 7900-8000, 7950-8050, 8000-8100, 8050-8150, 8100-8200, 8150-8250, 8200-8300, 8250-8350, 8300-8400, 8350-8450, 8400-8500, 8450-8550, 8500-8600, 8550-8650, 8600-8700, 8650-8750, 8700-8800, 8750-8850, 8800-8900, 8850-8950, 8900-9000, 8950-9050, 9000-9100, 9050-9150, 9100-9200, 9150-9250, 9200-9300, 9250-9350, 9300-9400, 9350-9450, 9400-9500, 9450-9550, 9500-9600, 9550-9650, 9600-9700, 9650-9750, 9700-9800, 9750-9850, 9800-9900, 9850-9950, 9900-10000, 9950-10050, 10000-10100, 10050-10150, 10100-10200, 10150-10250, 10200-10300, 10250-10350, 10300-10400, 10350-10450, 10400-10500, 10450-10550, 10500-10600, 10550-10650, 10600-10700, 10650-10750, 10700-10800, 10750-10850, 10800-10900, 10850-10950, 10900-11000, 10950-11050, 11000-11100, 11050-11150, 11100-11200, 11150-11250, 11200-11300, 11250-11350, 11300-11400, 11350-11450, 11400-11500, 11450-11550, 11500-11600, 11550-11650, 11600-11700, 11650-11750, 11700-11800, 11750-11850, 11800-11900, 11850-11950, 11900-12000, 11950-12050, 12000-12100, 12050-12150, 12100-12200, 12150-12250, 12200-12300, 12250-12350, 12300-12400, 12350-12450, 12400-12500, 12450-12550, 12500-12600, 12550-12650, 12600-12700, 12650-12750, 12700-12800, 12750-12850, 12800-12900, 12850-12950, 12900-13000, 12950-13050, 13000-13100, 13050-13150, 13100-13200, 13150-13250, 13200-13300, 13250-13350, 13300-13400, 13350-13450, 13400-13500, 13450-13550, 13500-13600, 13550-13650, 13600-13700, 13650-13750, 13700-13800, 13750-13850, 13800-13900, 13850-13950, 13900-14000, 13950-14050, 14000-14₁00, 14050-14150, 14100-14200, 14150-14250, 14200-14300, 14250-14350, 14300-14400, 14350-14450, 14400-14500, 14450-14550, 14500-14600, 14550-14650, 14600-14700, 14650-14750, 14700-14800, 14750-14850, 14800-14900, 14850-14950, 14900-14000, 15950-15050, 15000-15100, 15050-15150, 15100-15200, 15150-15250, 15200-15300, 15250-15350, 15300-15400, 15350-15450, 15400-15500, 15450-15550, 15500-15600, 15550-15650, 15600-15700, 15650-15750, 15700-15800, 15750-15850, 15800-15900, 15850-15950, 15900-14000, 16950-16050, 17000-17100, 17050-17150, 17100-17200, 17150-17250, 17200-17300, 17250-17350, 17300-17400, 17350-17450, 17400-17500, 17450-17550, 17500-17600, 17550-17650, 17600-17700, 17650-17750, 17700-17800; 17750-17850, 17800-17900, 17850-17950, 17900-14000, 17950-18050, 18000-18100, 18050-18150, 18100-18200, 18150-18250, 18200-183009 18250-18350, 18300-18400, 18350-18450, 18400-18500, 18450-18550, 18500-18600, 18550-18650, 18600-18700, 18650-18750, 18700-18800, 18750-18850, 18800-18900, 18850-18950, 18900-14000, 18950-19050, 19000-19100, 19050-19150, 19100-19200, 19150-19250, 19200-19300, 19250-19350, 19300-19400, 19350-19450, 19400-19500, 19450-19550, 19500-19600, 19550-19650, 19600-19700, 19650-19750, 19700-19800, 19750-19850, 19800-19900, 19850-19950, 19900-20000, 19950-20050, 20000-20100, 20050-20150, 20100-20200, 20150-20250, 20200-20300, 20250-20350, 20300-20400, 20350-20450, 20400-20500, 20450-20550, 20500-20600, 20550-20650, 20600-20700, 20650-20750, 20700-20800, 20750-20850, 20800-20900, 20850-20950, 20900-21000, 20950-21050, 21000-21100, 21050-21150, 21100-21200, 21150-21250, 21200-21300, 21250-21350, 21300-21400, 21350-21450, 21400-21500, 21450-21550, 21500-21600, 21550-21650, 21600-21700, 21650-21750, 21700-21800, 21750-21850, 21800-21900, 21850-21950, 21900-22000, 21950-22050, 22000-22100, 22050-22150, 22100-22200, 22150-22250, 22200-22300, 22250-22350, 22300-22400, 22350-22450, 22400-22500, 22450-22550, 22500-22600, 22550-22650, 22600-22700, 22650-22750, 22700-22800, 22750-22850, 22800-22900, 22850-22950, 22900-23000, 22950-23050, 23000-23100, 23050-23150, 23100-23200, 23150-23250, 23200-23300, 23250-23350, 23300-23400, 23350-23450, 23400-23500, 23450-23550, 23500-23600, 23550-23650, 23600-23700, 23650-23750, 23700-23800, 23750-23850, 23800-23900, 23850-23950, 23900-24000, 23950-24050, 24000-24100, 24050-24150, 24100-24200, 24150-24250, 24200-24300, 24250-24350, 24300-24400, 24350-24450, 24400-24500, 24450-24550, 24500-24600, 24550-24650, 24600-24700, 24650-24750, 24700-24800, 24750-24850, 24800-24900, 24850-24950, 24900-25000, 24950-25050, 25000-25100, 25050-25150, 25100-25200, 25150-25250, 25200-25300, 25250-25350, 25300-25400, 25350-25450, 25400-25500, 25450-25550, 25500-25600, 25550-25650, 25600-25700, 25650-25750, 25700-25800, 25750-25850, 25800-25900, 25850-25950, 25900-26000, 25950-26050, 26000-26100, 26050-26150, 26100-26200, 26150-26250, 26200-26300, 26250-26350, 26300-26400, 26350-26450, 26400-26500, 26450-26550, 26500-26600, 26550-26650, 26600-26700, 26650-26750, 26700-26800, 26750-26850, 26800-26900, 26850-26950, 26900-27000, 26950-27050, 27000-27100, 27050-27150, 27100-27200, 27150-27250, 27200-27300, 27250-27350, 27300-27400, 27350-27450, 27400-27500, 27450-27550, 27500-27600, 27550-27650, 27600-27700, 27650-27750, 27700-27800, 27750-27850, 27800-27900, 27850-27950, 27900-28000, 27950-28050, 28000-28100, 28050-28150, 28100-28200, 28150-28250, 28200-28300, 28250-28350, 28300-28400, 28350-28450, 28400-28500, 28450-28550, 28500-28600, 28550-28650, 28600-28700, 28650-28750, 28700-28800, 28750-28850, 28800-28900, 28850-28950, 28900-29000, 28950-29050, 29000-29100, 29050-29150, 29100-29200, 29150-29250, 29200-29300, 29250-29350, 29300-29400, 29350-29450, 29400-29500, 29450-29550, 29500-29600, 29550-29650, 29600-29700, 29650-29750, 29700-29800, 29750-29850, 29800-29900, 29850-29950, 29900-30000, 29950-30050, 30000-30100, 30050-30150, 30100-30200, 30150-30250, 30200-30300, 30250-30350, 30300-30400, 30350-30450, 30400-30500, 30450-30550, 30500-30600, 30550-30650, 30600-30700, 30650-30750, 30700-30800, 30750-30850, 30800-30900, 30850-30950, 30900-31000, 30950-31050, 31000-31100, 31050-31150,31100-31200, 31150-31250, 31200-31300, 31250-31350, 31300-31400, 31350-31450, 31400-31500, 31450-31550, 31500-31600, 31550-31650, 31600-31700, 31650-31750, 31700-31800, 31750-31850, 31800-31900, 31850-31950, 31900-32000, 31950-32050, 32000-32100, 32050-32150, 32100-32200, 32150-32250, 32200-32300, 32250-32350, 32300-32400, 32350-32450, 32400-32500, 32450-32550, 32500-32600, 32550-32650, 32600-32700, 32650-32750, 32700-32800, 32750-32850, 32800-32900, 32850-32950, 32900-33000, 32950-33050, 33000-33100, 33050-33150, 33100-33200, 33150-33250, 33200-33300, 33250-33350, 33300-33400, 33350-33450, 33400-33500, 33450-33550, 33500-33600, 33550-33650, 33600-33700, 33650-33750, 33700-33800, 33750-33850, 33800-33900, 33850-33950, 33900-34000, 33950-34050, 34000-34100, 34050-34150, 34100-34200, 34150-34250, 34200-34300, 34250-34350, 34300-34400, 34350-34450, 34400-34500, 34450-34550, 34500-34600, 34550-34650, 34600-34700, 34650-34750, 34700-34800, 34750-34850, 34800-34900, 34850-34950, 34900-35000, 34950-35050, 35000-35100, 35050-35150, 35100-35200, 35150-35250, 35200-35300, 35250-35350, 35300-35400, 35350-35450, 35400-35500, 35450-35550, 35500-35600, 35550-35650, 35600-35700, 35650-35750, 35700-35800, 35750-35850, 35800-35900, 35850-35950, 35900-36000, 35956-36050, 36000-36100, 36050-36150, 36100-36200, 36150-36250, 36200-36300, 36250-36350, 36300-36400, 36350-36450, 36400-36500, 36450-36550, 36500-36600, 36550-36650, 36600-36700, 36650-36750, 36700-36800, 36750-36850, 36800-36900, 36850-36950, 36900-37000, 36950-37050, 37000-37100, 37050-37150, 37100-37200, 37150-37250, 37200-37300, 37250-37350, 37300-37400, 37350-37450, 37400-37500, 37450-37550, 37500-37600, 37550-37650, 37600-37700, 37650-37750, 37700-37800, 37750-37850, 37800-37900, 37850-37950, 37900-38000, 37950-38050, 38000-38100, 38050-38150, 38100-38200, 38150-38250, 38200-38300, 38250-38350, 38300-38400, 38350-38450, 38400-38500, 38450-38550, 38500-38600, 38550-38650, 38600-38700, 38650-38750, 38700-38800, 38750-38850, 38800-38900, 38850-38950, 38900-39000, 38950-39050, 39000-39100, 39050-39150, 39100-39200, 39150-39250, 39200-39300, 39250-39350, 39300-39400, 39350-39450, 39400-39500, 39450-39550, 39500-39600, 39550-39650, 39600-39700, 39650-39750, 39700-39800, 39750-39850, 39800-39900, 39850-39950, 39900-40000, 39950-40050, 40000-40100, 40050-40150, 40100-40200, 40150-40250, 40200-40300, 40250-40350, 40300-40400, 40350-40450, 40400-40500, 40450-40550, 40500-40600, 40550-40650, 40600-40700, 40650-40750, 40700-40800, 40750-40850, 40800-40900, 40850-40950, 40900-41000, 40950-41050. Embodiments also exist in which PEG is in a larger range made by combining two or more of any of the foregoing ranges. The invention includes single PEG molecules as well as groups of PEG molecules. In embodiments involving a polydisperse plurality of PEG molecules, “molecular weight” for such pluralities refers to M_{W}.

TPGS analog compounds include compounds containing a tocopherol, a steroid or a flavonoid group. In some embodiments, the TPGS analog compound may be Chromanol-Succinate-PEG 1000, Chromanol-Succinate-PEG 400, PEG-r-PPG-970-BE-VitE succinate, PEG-b-PPG-b-PEG-1100-VitE succinate, PPG 1000-VitE succinate (TPPG 1000), BE-PPG-1000-VitE succinate, VitE-succinate-Oleate-860, Ibuprofen-PEG 1000, Indomethacin-PEG1000, Chromone-2-carboxylic acid-PEG 1000, Chromone-2-carboxylic acid-PEG 1100-OMe; Chromone-2-carboxylic acid-PEG 1500, Chromone-2-carboxylic acid-PEG 2000, Naproxen-PEG 1000, Probenecid-PEG 1000, Cholesterol-Succinate-PEG 1000, Cholesterol-Succinate-PEG 1100-OMe, Cholesterol-Succinate-PEG 1500, Cholesterol-Succinate-PEG 2000, 7-carboxymethoxy-4-methyl-coumarin-PEG 1000, 5-(4-chlorophenyl)-2-furoic acid-PEG 1000, Cholic acid-PEG 1000, Cholic acid-PEG 1100-OMe, Cholic acid-PEG 1500, Cholic acid-PEG 2000, Deoxy-cholic acid-PEG 1000, Probenecid-PEG 1000-Succinate-VitE, Lithocholic acid-PEG 1000, Mono-methyl-ether-PEG 1100-succinate-VitE, PEG 1500-succinate-VitE, Ursodeoxycholic acid-PEG 1000, Dehydrocholic acid-PEG 1000, Chenodeoxycholic acid-PEG 1000, Chromone-3-carboxylic acid-PEG 1000, 7-hydroxy-coumarinyl-4-acetic acid-PEG 1000, Tocopheryl-oxy-butyric acid-PEG 1000, Tocopheryl-oxy-acetic acid-PEG 1000, Vit E Succinate-glycerol propoxylate-PEG 1000, Vit E Succinate-glycerol propoxylate-PEG 1500, TPGS 750-OMe, TPGS 1500-OMe, TPGS 1500, TPGS 2000, TPGS 8000, (R)-(+)-6-hydroxy-2,5,7,8-tetramethyl, Chroman-2-carboxylic acid-PEG 1000, TPGS-2000-OMe, VitE-succinate-PEG-PPG-PEG-1900, VitE-succinate-PPG-PEG-MBE-1700, VitE-succinate-PPG 750, VitE-succinate-PPG 2000, VitE-succinate-PPG-PEG-PPG-2000, TPGS 8000 diester, Tri-VitE-succinate-glycerol proproxylate-1500, TPGS 14000, TPGS 20000, TPGS 14000 diester, TPGS 20000 diester, VitE-succinate-glycerol ethoxylate 1000, VitE-succinate-PPG 400, VitE-succinate-PPG 1200, Cholesterol-Succinate-PPG 1000, Stigmasterol-succinate-PEG 1000, gamma-TPGS 1000, gamma-VitE succinate PPG 1000 or Cholic acid-PPG 1000.

The chemical structures of representative TPGS compounds are illustrated in Table I.

Table I
Structures of Representative Compounds

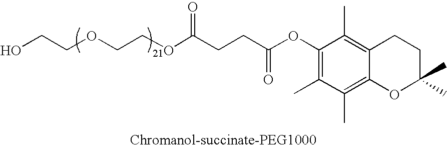

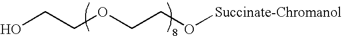

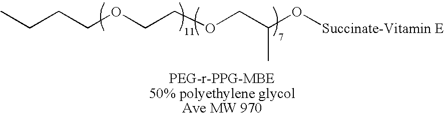

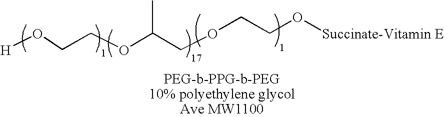

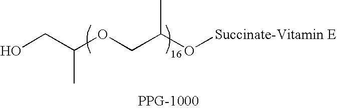

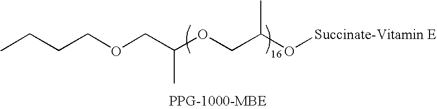

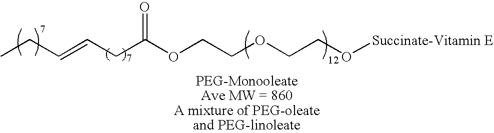

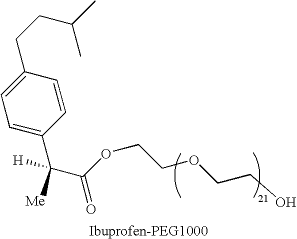

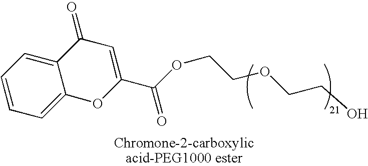

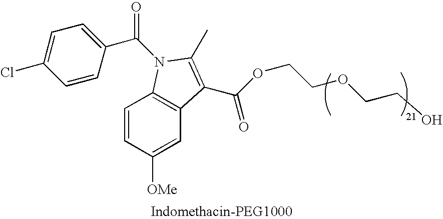

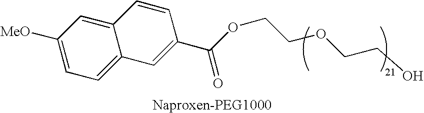

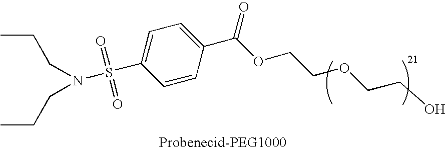

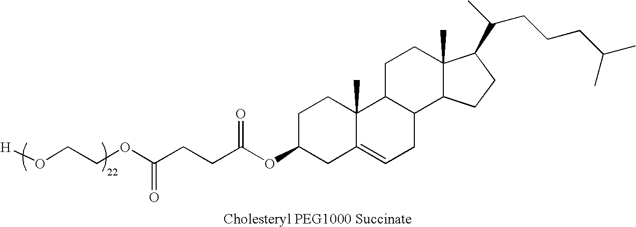

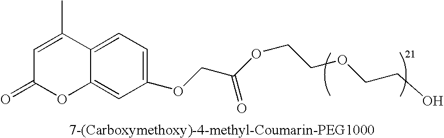

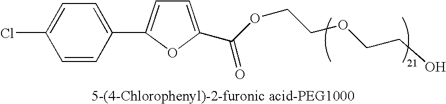

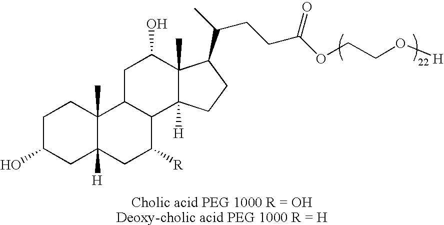

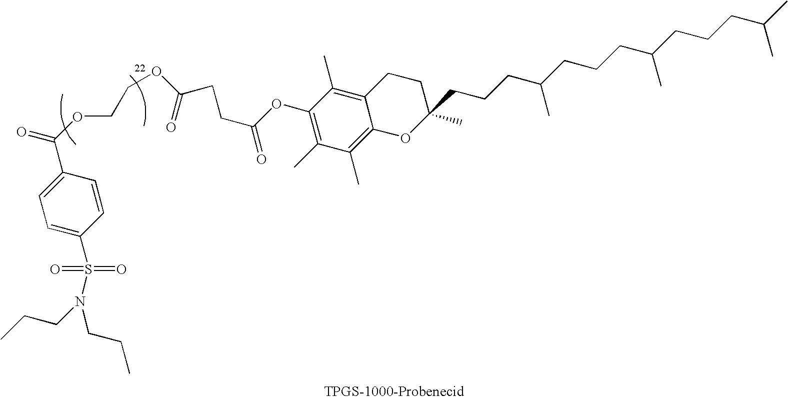

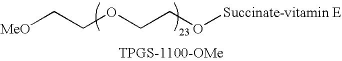

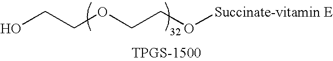

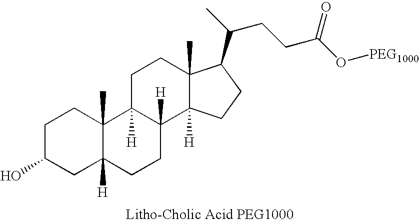

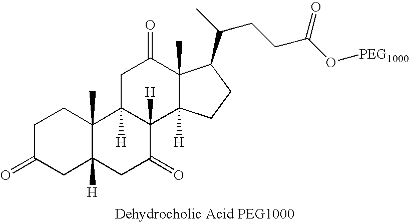

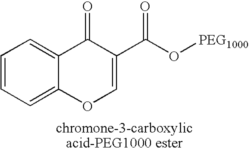

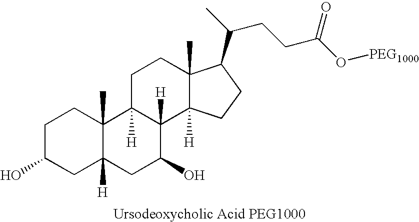

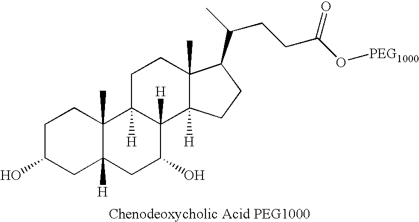

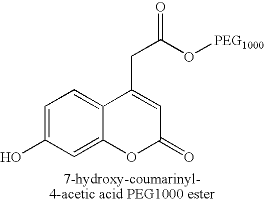

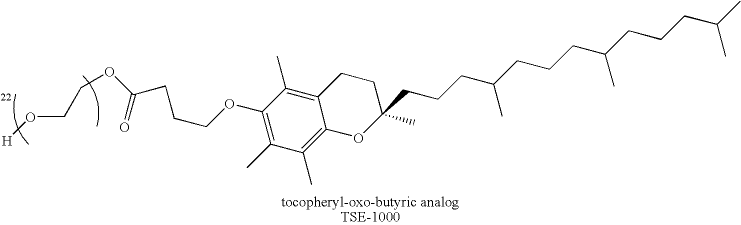

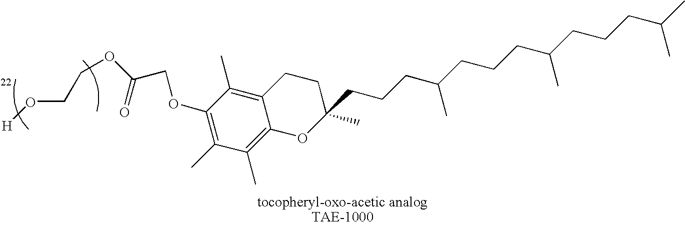

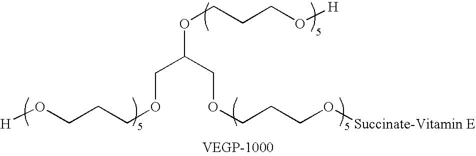

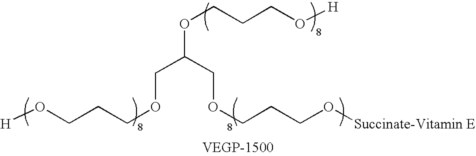

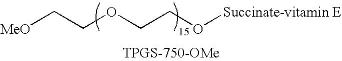

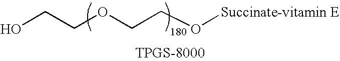

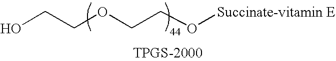

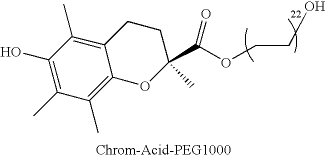

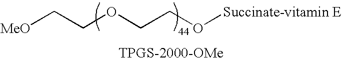

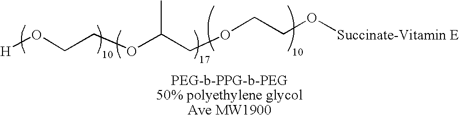

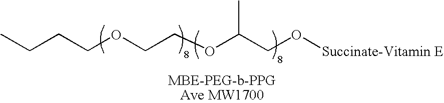

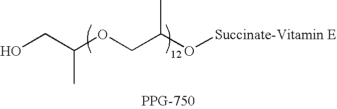

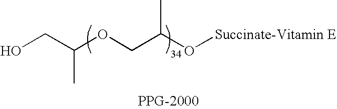

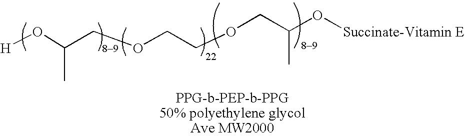

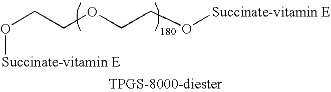

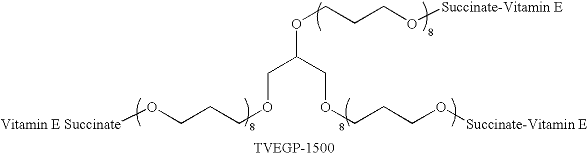

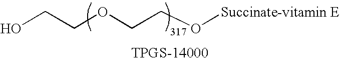

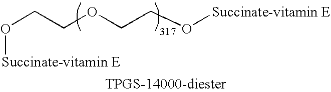

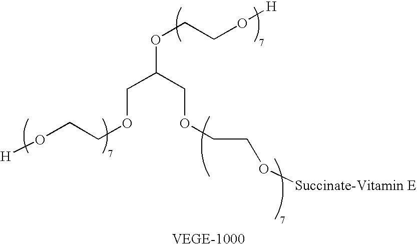

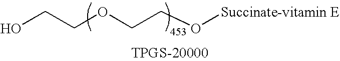

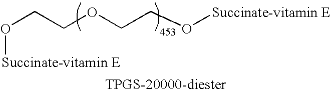

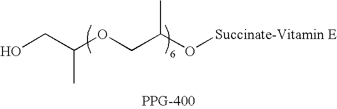

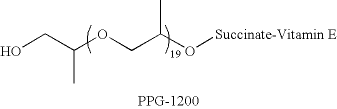

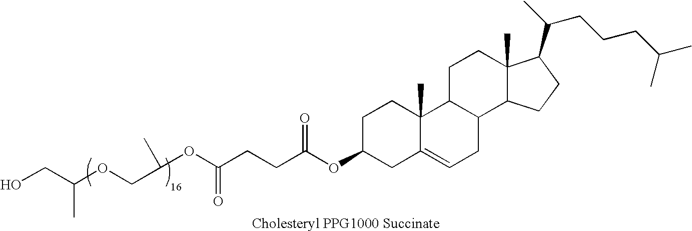

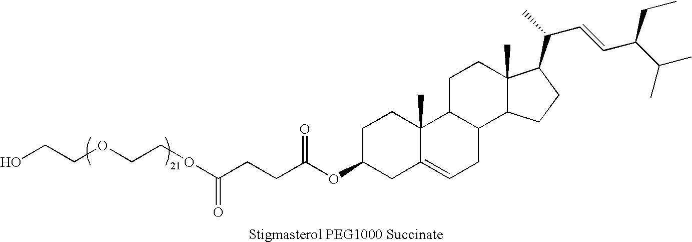

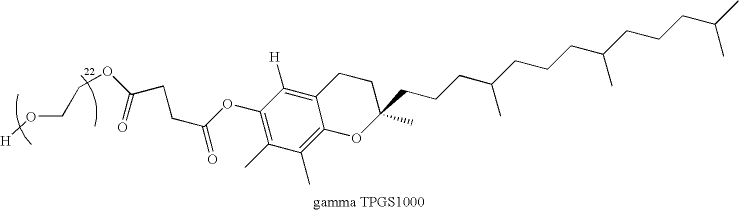

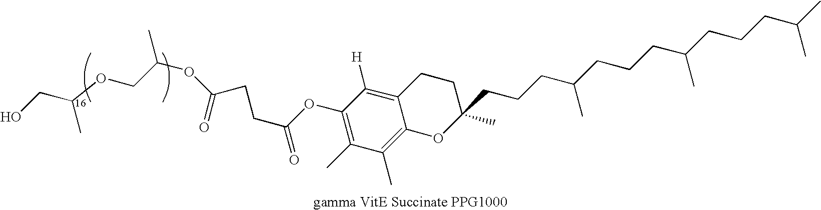

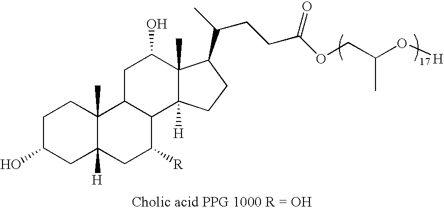

The invention also includes compositions that contain at least one pharmaceutical or lipophilic compound and at least one efflux inhibitor. Embodiments of such compositions exist involving all efflux inhibitors or pharmaceutical or lipophilic compounds described in this application as well as all combinations of such efflux inhibitors or pharmaceutical or lipophilic compounds. In some embodiments, the composition contains one or more pharmaceutical or lipophilic compounds along with an efflux inhibitor. In some embodiments, the pharmaceutical compound is a lipophilic compound for pharmaceutical use. In some embodiments, the compositions contain a pharmaceutically effective amount of a lipophilic compound for pharmaceutical use.

In some embodiments, the compositions of the present invention contain one or more additional desirable components or compounds. Any desirable compounds can be used. Examples include, but are not limited to, additional active pharmaceutical ingredients as well as excipients (e.g. cyclodextrins), diluents, and carriers such as fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; antioxidants; buffers; bacteriostats; coloring agents; perfuming agents; flavoring agents; lubricants (e.g., talc, calcium and magnesium stearate); solid polyethylene glycols; and mixtures. thereof. Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions.

It should be understood that the ingredients particularly mentioned above are merely examples and that some embodiments of formulations comprising the compositions of the present invention include other suitable components and agents. The compositions of the invention may be used for, among other things, pharmaceutical and cosmetic purposes and may be formulated with different ingredients according to the desired use.

Efflux inhibitors, pharmaceutical or lipophilic compounds and any additional components may be combined and formulated in any manner known in the pharmaceutical field.

The methods and compositions of the present invention may be used to increase the bioavailability of a pharmaceutical or lipophilic compound when an efflux inhibitor is administered to a subject before the pharmaceutical or lipophilic compound. In some embodiments, the efflux inhibitor may be administered separately from the pharmaceutical or lipophilic compound, for example, as separate compounds or compositions. In certain embodiments, the efflux inhibitor and pharmaceutical or lipophilic compound may be provided to a subject by different routes of administration.

The efflux inhibitor and the pharmaceutical or lipophilic compound may also be administered at the same time. This may be accomplished, for example, by administering them together as separate compounds or compositions or as one composition. In certain embodiments, a composition comprising an efflux inhibitor may be formulated to immediately release the efflux inhibitor when administered to the subject while the composition comprising a pharmaceutical or lipophilic compound may be formulated such that the pharmaceutical or lipophilic compound is released a suitable time after the efflux inhibitor is released. The composition comprising the efflux inhibitor and the composition comprising the pharmaceutical or lipophilic compound may then be administered to a subject at the same time.

A single composition comprising an efflux inhibitor and a pharmaceutical or lipophilic compound wherein the efflux inhibitor is released before the pharmaceutical or lipophilic compound may also be administered to a subject. Methods of formulating compositions wherein one component is released immediately and another component is released after the first component are known in the pharmaceutical arts. For example, U.S. Pat. Nos. 5,474,786; 6,183,778 and 6,908,626 each disclose compositions with a combination of immediate release and controlled release characteristics.

In certain embodiments, the efflux inhibitor may be administered to a subject an effective time prior to administration of the pharmaceutical or lipophilic compound. In other embodiments, the efflux inhibitor may be allowed to contact the target cells or tissues an effective time prior to contacting the target cells or tissues with the pharmaceutical or lipophilic compound. Effective times can range from less than one minute to several hours, for example, from 15 minutes to 45 minutes. In some embodiments, the effective time may be from 1 minute to 15 minutes, from 15 minutes to 30 minutes, from 30 minutes to 45 minutes, from 45 minutes to one hour, from one hour to 1.5 hours, from 1.5 hours to 2 hours, from 2 hours to 3 hours, from 3 hours to 4 hours and from 4 hours to six hours. In certain embodiments, the effective time can be greater than 6 hours. Examples of effective times include, but are not limited to, one minute, 5 minutes, 10 minutes, 15 minutes, 20 minutes, 30 minutes, 45 minutes, one hour, 1.5 hours, 2 hours, 3 hours and 4 hours. Formulating the efflux inhibitor and the pharmaceutical or lipophilic compound so that the efflux inhibitor releases before the pharmaceutical or lipophilic compound at any of these times or ranges of times is within the knowledge of those skilled in the art.

One of skill in the art may also determine an increase in the bioavailability of a pharmaceutical or lipophilic compound using assays standard in the art. For example, the plasma or tissue concentration of a pharmaceutical or lipophilic compound in an animal may be determined after administration of the pharmaceutical or lipophilic compound alone or after the administration of the pharmaceutical or lipophilic compound at the same time as the efflux inhibitor or after administration of an efflux inhibitor followed by the administration of the pharmaceutical or lipophilic compound. In addition, in vitro assays such as the Caco-2 cell transport assay described below can be used to assess absorptive and secretory transport of a pharmaceutical or lipophilic compound across a cell monolayer, thereby providing an indication of the bioavailability of a pharmaceutical or compound when administered to a subject.

Increases in bioavailability may be determined by measuring the plasma concentration of a pharmaceutical or lipophilic compound in an animal after dosing with a pharmaceutical or lipophilic compound in the presence or absence of an efflux inhibitor or with or without prior administration of an efflux inhibitor. AUC values may be determined and compared to calculate the increase in bioavailability. In some embodiments, the methods or compositions of the invention may increase the bioavailability of a pharmaceutical or lipophilic compound by a factor of 0.1 to 10 in comparison to the pharmaceutical or lipophilic compound alone. In other embodiments, the methods or compositions of the invention may increase the bioavailability of a pharmaceutical or lipophilic compound by a factor of 0.1 to 10 in comparison to the pharmaceutical or lipophilic compound administered at the same time as the efflux inhibitor. In certain embodiments, the increase may be by a factor of 0.1 to 1, 1 to 2, 2 to 3, 3 to 4, 4 to 5, 5 to 6, 6 to 7, 7 to 8, 8 to 9 or 9 to 10.

In certain embodiments, the methods and compositions of the present invention may be used to increase the oral bioavailability of a pharmaceutical or lipophilic compound. The compounds and compositions of the invention may be administered to a subject so that the efflux inhibitor is released into the gut of a subject before the pharmaceutical or lipophilic compound.

The compositions of the invention may also be administered to a subject to inhibit efflux. Efflux inhibition may be determined using standard assays such as the Caco-2 cell assay disclosed below. One of skill in the art will recognize that standard assays may also be used to predict efflux inhibition for a composition of the present invention to be administered to a subject such as a human.

Percentage of efflux inhibition may be determined by comparing the amount of a compound (such as Rhodamine 123) transported across a Caco-2 cell monolayer in the presence or absence of a TPGS compound. Percentage of efflux inhibition values may be calculated by determining the ratio (efflux ratio) of Rhodamine 123 permeability in the basolateral to apical (Bl-Ap) direction to Rhodamine 123 permeability in the apical to basolateral (Ap-Bl) direction in the presence or absence of an efflux inhibitor. In some embodiments, the compounds or compositions of the invention may inhibit efflux by 1-10%; in other embodiments, by 10-20%, 20-30%,30-40%,40-50%, 50-60%, 60-70%, 70-80%, 80-90% or 90-100%. In certain embodiments, the compounds or compositions of the invention may inhibit efflux by greater than 75%; in other embodiments, by greater than 85%; in still other embodiments, by greater than 95%.

The compounds or compositions of the invention may be used in any amount effective for efflux inhibition or to increase the bioavailability of a pharmaceutical or lipophilic compound. One of skill in the art will recognize that effective amounts may vary depending upon, among other variables, the efflux inhibitor utilized, the nature of the pharmaceutical or lipophilic compound, any additional components present in the composition, the size of the patient, the dosage form, the route of administration, and the like. The effective amount of an efflux inhibitor or efflux inhibitor present in a composition of the invention may be routinely determined by one of skill in the art using standard in vitro and in vivo assays described herein. For example, animal studies may be used to determine the range of effective amounts and these data may be extrapolated to determine an effective amount for administration to a human.

The effective amount of an efflux inhibitor or efflux inhibitor present in a composition of the invention may range from about 0.1 to 100 milligrams (mg) per kilogram (kg) of subject weight. In certain embodiments, the compounds or compositions of the invention are administered at from about 0.1 mg/kg to 2 mg/kg or from about 2 mg/kg to 5 mg/kg; in other embodiments, from about 5 mg/kg to 10 mg/kg, from about 10 mg/kg to 20 mg/kg, from about 20 mg/kg to 30 mg/kg, from about 30 mg/kg to 40 mg/kg, from about 40 mg/kg to 50 mg/kg, from about 50 mg/kg to 75 mg/kg or from about 75 mg/kg to 100 mg/kg.

Compositions of the invention containing a pharmaceutical compound may be administered to a subject to treat or prevent a disease or disorder treatable by the pharmaceutical compound. Administration of compositions of the invention containing a pharmaceutical compound may increase the amount of the pharmaceutical compound in the plasma or tissue of a subject. One of skill in the art will recognize that the amount of pharmaceutical compound present in the composition may have to be altered accordingly to provide the desired effective amount of the pharmaceutical compound.

In some embodiments, the compounds or compositions of the present invention may be administered to a subject. Suitable subjects include a cell, population of cells, tissue or organism. In certain embodiments, the subject is a mammal such as a human. The compounds or compositions may be administered in vitro or in vivo.

In some embodiments, the compounds or compositions of the present invention are administered to persons or animals to provide substances in any dose range that will produce desired physiological or pharmacological results. Dosage will depend upon the substance or substances administered, the therapeutic endpoint desired, the desired effective concentration at the site of action or in a body fluid, and the type of administration. Information regarding appropriate doses of substances are known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn., (6.sup.th Ed. 1995).

The compounds or compositions can be administered in any form by any means. Examples of forms of administration include, but are not limited to, injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art. In some embodiments, the compositions can be combined with other components. Examples include, but are not limited to, coatings, depots, matrices for time release and osmotic pump components.

Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration (for example, at the location of a tumor or internal injury), administration into the lumen or parenchyma of an organ, and parenteral administration.

The invention further includes packages, vessels, kits, or any other type of container that contains compositions of the present invention or any compound or composition suitable for use with the methods of the present invention. The package, vessel or container contains, is labeled with, or is otherwise accompanied by instructions to use the compound or composition to enhance or increase the bioavailability of at least one pharmaceutical or lipophilic compounds when administered to a subject.

The present invention also includes compositions, articles, and kits. Single compositions or articles of the type described above are within the present invention. In addition, the invention comprises kits for administration of at least one efflux inhibitor and at least one pharmaceutical or lipophilic compound. In some embodiments, the at least one efflux inhibitor is present in a first composition and the at least one pharmaceutical or lipophilic compound is present in a second composition such that the two compositions are to be administered either at the same or different times or via the same or different routes. The kit optionally includes instructions specifying differences in timing of administration, means of administration, or both. Kits specifying any of the differences in time or administration specified above are within the invention.

The following are examples of methods that can be used to produce efflux inhibitors for use in the methods of the present invention.

Materials: non-essential amino acids, streptomycin and penicillin, rhodamine 123 (RHO), methanol, acetonitrile, dichloromethane, iso-propyl alcohol, water, formic acid, ammonium acetate, sodium hydroxide, PEG750-OMe, PEG 1000, PEG1100-OMe, PEG1500, PEG2000, DMAP (4-dimethylaminopyridine), DCC (N,N′-dicyclohexylcarbodiimide), N-2-hydroxyethylpiperazine-N′-2-ethane sulfonic acid (HEPES), and Dulbecco's modified Eagle medium (DMEM), sodium chloride, potassium chloride, D-glucose, calcium chloride, magnesium chloride, and potassium hydrogenphosphate trihydrate were purchased from Sigma-Aldrich (St. Louis, Mo.). Heat-inactivated fetal calf serum (FBS) and Hank's Buffered Saline Solution (HBSS) were purchased from GIBCO (Invitrogen Corp.; Carlsbad, Calif.). Materials were stored as suggested by their manufacturer.

General synthetic procedure: vitamin E succinate (3.25 g, 6.12 mmol) was dissolved in dichloromethane (20 mL) and 1.1 equivalents of the corresponding polyethylene glycol added and stirred at room temperature. DMAP (0.1 equivalents) and DCC (1.1 equivalents) were added sequentially. The reaction vessel was capped and stirred overnight. The reaction mixture was filtered through a Buchner funnel, and the filtrate concentrated under reduced pressure to afford crude product(s). Products (TPGS750-OMe, TPGS 1000, TPGS 1000 diester, TPGS 1100-OMe, TPGS 1500, TPGS 1500 diester, TPGS2000 and TPGS2000 diester) were then purified via preparative HPLC (Dynamax Microsorb C8, 250×41.4 mm I.d., 8μ particles, 60 Å pore) using mobile phases (A, 25/75 methanol/acetonitrile (ACN); B, 25/75 iso-propyl alcohol (IPA)/ACN; and C, IPA) with general gradient conditions of A for 24 min, B for 6 min and C for 12 min at a flow rate of ˜80 mL/min. TPGS1000; ¹³C-NMR (125 MHz, CDCl₃): 10.8 (—CH₃), 11.0 (—CH₃), 11.9 (—CH₃), 18.8 (—CH₃), 18.9 (—CH₃), 19.7 (—CH₂), 20.1 (—CH₂), 21.7 (—CH₃), 22.9 (—CH₃), 23.5 (—CH₂), 23.8 (—CH₂), 27.0 (—CH), 27.9 (—CH₂), 28.2 (—CH₂), 30.5 (—CH₂), 31.7 (—CH), 31.8 (—CH), 36.4 (—CH₂), 36.5 (—CH₂), 36.5 (—CH₂), 36.6 (—CH₂), 38.5 (—CH₂), 39.3 (—CH₂), 60.6 (—CH₂), 62.9 (—CH₂), 68.1* (—CH₂), 69.6* (—CH₂), 69.8* (—CH₂), 71.9* (—CH₂), 74.0 (—C), 116.3 (—C), 121.7 (—C), 124.1 (—C), 125.8 (—C), 140.0 (—C), 148.4 (—C), 169.5 (—C), and 170.8 (—C); * represents the polyethylene glycol carbons.

The following are examples of in vitro assays that can be used to evaluate the efflux inhibition or bioavailability properties of compositions of the present invention.

Caco-2 cell culture and handling: Caco-2 cells, clone C2BBe1, were purchased from American Type Culture Collection (ATCC; Manassas, Va.) and used from passage 70-92 with Corning Incorp. Life Sciences (Acton, Mass.) polycarbonate membranes (3460; 12-well, pore size 0.4 μm, 1.13 cm²). Cells were seeded at a density of ˜60,000 cells/cm² and grown at ˜37° C. in a controlled atmosphere of ˜5% CO₂ with a relative humidity of ˜85%. The culture medium consisted of DMEM supplemented with 10% FBS, 1% non-essential amino acids, 100 μg/mL streptomycin and 100 U/mL penicillin. Transepithelial electrical resistance (TEER) was measured with a hand-held REMS electrical volt-ohm meter EVOM (World Precision Instruments; Sarasota, Fla.). Only monolayers with a TEER >350 Ω*cm², with background subtracted, were used for transport studies.

Using the transport assay, drug transport was assessed in absorptive (apical to basolateral, Ap→Bl) and secretory (Bl→Ap) directions. Prior to the RHO transport experiments, unless otherwise denoted, the monolayers were pre-incubated for ˜1 h with or without TPGS1000. Subsequently, at t=0 min, a solution of RHO (13 μM) in buffer solution (pH ˜7.4) was added to the donor compartment and pure buffer solution (freshly prepared Krebs Ringer Buffer (KRB) at pH ˜7.4 on both the apical and basolateral sides) added to the receiver compartment. In some experiments, both sides contained TPGS 1000 while other experiments had TPGS 1000 present on the receiver or donor side only.

Cell monolayers were agitated using an orbital shaker (IKA®—Werke GmbH & CO KG; Staufen, Germany) at 100±20 rpm. Samples were collected after 30, 60, 120, 180, 240, and 300 min from the receiver compartment. After each sampling, an equal volume of fresh transport buffer (˜37° C.) was added to the receiver compartment. RHO was quantified with a Cytofluor-2 fluorescence plate reader (Perkin Elmer Biosystems; Weiterstadt, Germany) operating at excitation wavelength of 485 nm and emission wavelength of 530 nm to measure RHO.

Apparent Permeability and statistical analysis: flux was determined using receiver compartment RHO steady-state appearance rates (ΔQ/Δt). Pₐₚₚ across Caco-2 monolayers was calculated via:

$${P}_{app} = \frac{Δ Q}{Δ t \cdot A \cdot {C}_{0}}$$

Pₐₚₚ is apparent permeability coefficient [cm/s], ΔQ/Δt is permeability rate [μg/s; pmol/s], C₀ is initial concentration in donor chamber [μg/cm³; pmol/cm³], and A is membrane surface area [cm²]. Pₐₚₚ Ap→Bl, Pₐₚₚ Bl→Ap, and active transport are expressed as means ± standard deviation (SD) or as standard error of the mean (SEM). Efflux Ratio (ER) was computed as ER=(Pₐₚₚ Bl→Ap)/(Pₐₚₚ Ap→Bl).

Graphs were prepared using Prism 4.02™ (GraphPad Software, Inc.; San Diego, Calif.). Active transport Kₘ was computed from non-linear regression analysis (hyperbola).

The experimental results presented in FIG. 1A demonstrate how the pre-exposure of Caco-2 cell monolayers to TPGS 1000 effectively increases the amount of compound transported across the cells (apical to basolateral) in a time-dependent fashion. For example, FIG. 1A shows that a 45 minute preincubation resulted in a greater than two-fold increase in Pₐₚₚ compared to that achieved without TPGS 1000 pre-exposure at the 120 minute time point. A higher in vitro Pₐₚₚ equates to more compound being transported to the basolateral side of the cell monolayer, which in turn equates with a higher in vivo bioavailability (AUC).

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. A method of increasing the bioavailability of at least one pharmaceutical compound comprising administering an effective amount of at least one efflux inhibitor to a subject before administering the at least one pharmaceutical compound to the subject.

2. The method of claim 1, wherein the subject is a mammal.

3. The method of claim 2, wherein the subject is a human.

4. The method of claim 1, wherein the at least one efflux inhibitor is administered to the subject at least 15 minutes before administering the at least one pharmaceutical compound to the subject.

5. The method of claim 1, wherein the at least one efflux inhibitor is administered to the subject at least 30 minutes before administering the at least one pharmaceutical compound to the subject.

6. The method of claim 1, wherein the at least one efflux inhibitor is administered to the subject at least 45 minutes before administering the at least one pharmaceutical compound to the subject.

7. The method of claim 1, wherein the at least one efflux inhibitor is administered to the subject at least one hour before administering the at least one pharmaceutical compound to the subject.

8. The method of claim 1, wherein the at least one efflux inhibitor and at least one pharmaceutical compound are administered orally.

9. The method of claim 1, wherein the at least one pharmaceutical compound is at least one lipophilic compound.

10. The method of claim 1, wherein the at least one efflux inhibitor is at least one TPGS compound.

11. The method of claim 10, wherein the at least one TPGS compound is selected from TPGS 1000, TPGS 1100-OMe, TPGS 1500, TPGS 2000, PEG-r-PPG-970-BE-VitE succinate, PEG-b-PPG-b-PEG-1100-VitE succinate, PPG 1000-VitE succinate (TPPG 1000), Chromone-2-carboxylic acid-PEG 1000, Chromone-2-carboxylic acid-PEG 1100-OMe, Chromone-2-carboxylic acid-PEG 1500, Chromone-2-carboxylic acid-PEG 2000, Cholesterol-Succinate-PEG 1000, Cholesterol-Succinate-PEG 1100-OMe, Cholesterol-Succinate-PEG 1500, Cholesterol-Succinate-PEG 2000, Cholic acid-PEG 1000, Cholic acid-PEG 1100-OMe, Cholic acid-PEG 1500 and Cholic acid-PEG 2000.

12. A method of formulating a composition comprising combining at least one efflux inhibitor and at least one pharmaceutical compound, wherein the at least one efflux inhibitor is released from the composition before the at least one pharmaceutical compound when the composition is administered to a subject.

13. The method of claim 12, wherein the composition releases the at least one efflux inhibitor at least 15 minutes before the at least one pharmaceutical compound when the composition is administered to a subject.

14. The method of claim 12, wherein the composition releases the at least one efflux inhibitor at least 30 minutes before the at least one pharmaceutical compound when the composition is administered to a subject.

15. The method of claim 12, wherein the composition releases the at least one efflux inhibitor at least 45 minutes before the at least one pharmaceutical compound when the composition is administered to a subject.

16. The method of claim 12, wherein the composition releases the at least one efflux inhibitor at least one hour before the at least one pharmaceutical compound when the composition is administered to a subject.

17. The method of claim 12, wherein the composition is formulated for oral administration to a subject.

18. The method of claim 12, wherein the at least one pharmaceutical compound is at least one lipophilic compound.

19. The method of claim 12, wherein the at least one efflux inhibitor is at least one TPGS compound.

20. The method of claim 19, wherein the at least one TPGS compound is selected from TPGS 1000, TPGS 1100-OMe, TPGS 1500, TPGS 2000, PEG-r-PPG-970-BE-VitE succinate, PEG-b-PPG-b-PEG-1100-VitE succinate, PPG 1000-VitE succinate (TPPG 1000), Chromone-2-carboxylic acid-PEG 1000, Chromone-2-carboxylic acid-PEG 1100-OMe, Chromone-2-carboxylic acid-PEG 1500, Chromone-2-carboxylic acid-PEG 2000, Cholesterol-Succinate-PEG 1000, Cholesterol-Succinate-PEG 1100-OMe, Cholesterol-Succinate-PEG 1500, Cholesterol-Succinate-PEG 2000, Cholic acid-PEG 1000, Cholic acid-PEG 1100-OMe, Cholic acid-PEG 1500 and Cholic acid-PEG 2000.

21. A composition comprising:
(a) at least one efflux inhibitor; and
(b) at least one pharmaceutical compound;
wherein the composition is capable of releasing the at least one efflux inhibitor before the at least one pharmaceutical compound when the composition is administered to a subject.

22. The composition of claim 21, wherein the composition releases the at least one efflux inhibitor at least 15 minutes before the at least one pharmaceutical compound when the composition is administered to a subject.

23. The composition of claim 21, wherein the composition releases the at least one efflux inhibitor at least 30 minutes before the at least one pharmaceutical compound when the composition is administered to a subject.

24. The composition of claim 21, wherein the composition releases the at least one efflux inhibitor at least 45 minutes before the at least one pharmaceutical compound when the composition is administered to a subject.

25. The composition of claim 21, wherein the composition releases the at least one efflux inhibitor at least one hour before the at least one pharmaceutical compound when the composition is administered to a subject.

26. The composition of claim 21, wherein the composition is formulated for oral administration to a subject.

27. The composition of claim 21, wherein the at least one pharmaceutical compound is at least one lipophilic compound.

28. The composition of claim 21, wherein the composition releases the at least one efflux inhibitor and the at least one pharmaceutical compound in the gut of the subject.

29. The composition of claim 28, wherein the at least one efflux inhibitor is at least one TPGS compound.

30. The composition of claim 29, wherein the at least one TPGS compound is selected from TPGS 1000, TPGS 1100-OMe, TPGS 1500, TPGS 2000, PEG-r-PPG-970-BE-VitE succinate, PEG-b-PPG-b-PEG-1100-VitE succinate, PPG 1000-VitE succinate (TPPG 1000), Chromone-2-carboxylic acid-PEG 1000, Chromone-2-carboxylic acid-PEG 1100-OMe, Chromone-2-carboxylic acid-PEG 1500, Chromone-2-carboxylic acid-PEG 2000, Cholesterol-Succinate-PEG 1000, Cholesterol-Succinate-PEG 1100-OMe, Cholesterol-Succinate-PEG 1500, Cholesterol-Succinate-PEG 2000, Cholic acid-PEG 1000, Cholic acid-PEG 1100-OMe, Cholic acid-PEG 1500 and Cholic acid-PEG 2000.

31. A method of treating a subject in need of treatment with at least one pharmaceutical compound comprising administering an effective amount of at least one efflux inhibitor to the subject before administering the at least one pharmaceutical compound to the subject.

32. The method of claim 31, wherein the subject is a mammal.

33. The method of claim 32, wherein the subject is a human.

34. The method of claim 31, wherein the at least one efflux inhibitor is administered to the subject at least 15 minutes before administering the at least one pharmaceutical compound to the subject.

35. The method of claim 31, wherein the at least one efflux inhibitor is administered to the subject at least 30 minutes before administering the at least one pharmaceutical compound to the subject.

36. The method of claim 31, wherein the at least one efflux inhibitor is administered to the subject at least 45 minutes before administering the at least one pharmaceutical compound to the subject.

37. The method of claim 31, wherein the at least one efflux inhibitor is administered to the subject at least one hour before administering the at least one pharmaceutical compound to the subject.

38. The method of claim 31, wherein the at least one efflux inhibitor and the at least one pharmaceutical compound are administered orally.

39. The method of claim 31, wherein the at least one efflux inhibitor is at least one TPGS compound.

40. The method of claim 39, wherein the at least one TPGS compound is selected from TPGS 1000, TPGS 1100-OMe, TPGS 1500, TPGS 2000, PEG-r-PPG-970-BE-VitE succinate, PEG-b-PPG-b-PEG-1100-VitE succinate, PPG 1000-VitE succinate (TPPG 1000), Chromone-2-carboxylic acid-PEG 1000, Chromone-2-carboxylic acid-PEG 1100-OMe, Chromone-2-carboxylic acid-PEG 1500, Chromone-2-carboxylic acid-PEG 2000, Cholesterol-Succinate-PEG 1000, Cholesterol-Succinate-PEG 1100-OMe, Cholesterol-Succinate-PEG 1500, Cholesterol-Succinate-PEG 2000, Cholic acid-PEG 1000, Cholic acid-PEG 1100-OMe, Cholic acid-PEG 1500 and Cholic acid-PEG 2000.

41. A method of treating a subject in need of treatment with at least one pharmaceutical compound comprising administering an effective amount of the composition of claim 21 to the subject.

42. The method according to claim 41, wherein the composition is administered orally.

43. The method of claim 41, wherein the subject is a mammal.

44. The method of claim 43, wherein the subject is a human.

45. An article comprising:
a composition comprising at least one efflux inhibitor, and
a composition comprising at least one pharmaceutical compound
wherein, upon oral administration to a human or mammal, the article releases the at least one efflux inhibitor into the gut of the human or mammal before the composition releases the at least one pharmaceutical compound into the gut of the human or mammal.

46. A kit comprising:
a composition comprising at least one efflux inhibitor,
a composition comprising at least one pharmaceutical compound, and
instructions for administration of both the composition comprising at least one efflux inhibitor and the composition comprising at least one pharmaceutical compound to a human or mammal in a manner that will result in the at least one efflux inhibitor contacting selected cells, tissues or organs for a selected period of time before the at least one pharmaceutical compound contacts the selected cells, tissues or organs.

47. A container, wherein the contents of the container comprise;
at least one pharmaceutical compound, and
at least one efflux inhibitor,
wherein the container contains, is labeled, or is otherwise accompanied by instructions for administration of both the at least one efflux inhibitor and the at least one pharmaceutical compound to a human or mammal in a manner that will result in the at least one efflux inhibitor contacting selected cells, tissues or organs for a selected period of time before the at least one pharmaceutical compound contacts the selected cells, tissues or organs.
